(19) United States
(12) Patent Application Publication
(10) Pub. No.: US 2005/0131526 A1
(43) Pub. Date: Jun. 16, 2005

(54) Stent and balloon system for bifurcated vessels and lesions

(76) Inventor: Shing-Chiu Wong, Manhasset, NY (US)

Correspondence Address: Leopold Presser;Scully, Scott, Murphy & Presser, 400 Garden City Plaza, Garden City, NY, 11530, US

(21) Appl. No.: 10/999,706

(22) Filed: Nov. 30, 2004

Related U.S. Application Data
(60) Provisional application No. 60/528,315, filed on Dec. 10, 2003.

(51) Int. Cl. A61F 2/06

(52) U.S. Cl. 623/1.15

(57) ABSTRACT

A stent and a percutaneous balloon system for the placement of the stent in the lumen of a patient, and particularly relates to a stent and balloon system, which may be an integrated delivery system for dilatation and/or placement of a stent or stents especially for but not limited to bifurcated vessels and lesions. Moreover, disclosed is a method of deploying the stent at a particular site through the intermediary of the inventive balloon system.

### REFERENCE TO RELATED APPLICATION

The present application claims the benefit of the filing date of Dec. 10, 2003 of U.S. Provisional Patent Application No. 60/528,315.

### BACKGROUND OF THE INVENTION

1. Field of the Invention

The present invention relates to a stent and to a percutaneous balloon system facilitating the placement of the stent in the lumen of a patient. More particularly, the invention pertains to a stent and balloon system, which may constitute an integrated delivery system for dilatation and/or the placement of a stent or of stents, which is especially intended for, but not limited to applications in connection with bifurcated vessels and lesions. Moreover, the invention is also directed to the provision of a novel method of deploying the stent at a particular site through the intermediary of the inventive balloon system.

In essence, the medical technology has at this time been extensively developed with regard to the concepts of developing the delivery and deployment of diverse types of luminal stents and stent positioning devices, such as balloons for inserting and positioning such luminal stents. In particular, in the current medical technology stents are extensively employed in connection with implementing procedures, such as balloon angioplasty, and are effectively employed in the treatment of coronary artery disease.

Furthermore, although the advances in the placement of stents while maintaining the patency or integrity of the body lumen or vessel of a patient in the treatment of coronary obstructions represent a significant breakthrough in interventional cardiology, at present there is no effective treatment available through a dedicated percutaneous treatment system which is able to implement the treating of and obviating the problems which are encountered in the presence of particularly challenging lesion subsets.

Concerning the foregoing aspects, it is essential that a number of challenges must be currently considered in the conceptualizing or designing of a dedicated system adapted for the treatment of bifurcated lesions which occur in the body vessels or lumen of a patient, and which reflect various medical and technological viewpoints in the design and methods of use of such a treatment system, the latter of which may be either deemed a separate stent and balloon design, or the provision thereof as a dedicated or integrated treatment system.

In particular, various important and diverse criteria must be met in providing a dedicated system for the treatment of bifurcated lesions, which are encountered in the body vessels or lumen of a patient. These criteria can essentially be enumerated as follows:
1) A stent and balloon deployment system that is not cumbersome in the use thereof by a physician or surgeon.
2) A system that is compatible with a wide range of guide catheter sizes.
3) A system that is sufficiently flexible to be able to accommodate any size of combinations of main and side branches of a lumen or body vessel of a patient.
4) A system that is sufficiently flexible to be adapted for any angular combinations, which are present between main and side branches of the lumen or body vessels of a patient.
5) A system which is capable of minimizing any danger of encountered wire entanglement or crossing during deployment prior to reaching the site of the branch point.
6) Providing an inventory of stent and balloon systems adapted for large size combinations of lumen side and main branches, which is maintained within reasonable and economical bounds.
7) In the sphere of drug eluting stents, providing a design that would maximize vessel/stent strut contact for optimal drug delivery to the patient.
8) A stent and balloon system design that would not require the deployment of any advanced or technically demanding new implantation techniques, but is compatible with currently available technologies.

2. Discussion of the Prior Art

Although numerous patents and publications are currently in existence, which are directed to the disclosures of diverse types of luminal stents and delivery systems, including devices such as balloons employed in angioplasty for inserting and locating the luminal stents at specified sites and thereafter fixed in the patient's vessel, for instance, as in a blood vessel or other locales, these are not specifically designed and generally not suited for the treatment of bifurcated vessels and lesions.

Igaki, U.S. Pat. No. 5,762,625, discloses a luminal stent which is adapted to be introduced into a blood vessel, lymph vessel, bile duct, ureter, or esophagus among others for maintaining the shape or patency of the vessel, and wherein a balloon delivery arrangement may be employed for positioning and fixing the stent at a particular site. However, in this patent, there is no capability or concept of employing the stent and balloon delivery system for the treatment of bifurcated vessels and lesions in a manner analogous to that provided by the present invention.

Bosley, Jr., U.S. Pat. No. 5,514,176 discloses a pull apart coil stent which is adapted to be deployed in a body lumen, such as a urethra, ureters, common bile duct, vagina, cervix, fallopian tubes, sinus tract, rectum, bowel, esophagus or in the vascular system of a patient and also describes structure whereby the configuration of the coil may be varied in order to enable the positioning and any required removal thereof. This may be implemented through the intermediary of a suitable balloon catheter, for example, of the type described in the Palmas, U.S. Pat. No. 4,739,762, or the Giantorko, U.S. Pat. Nos. 4,580,568 and 4,907,336. None of these patents are adapted to provide nor do they disclose treatment systems employing the unique stent and balloon delivery arrangements of the invention for the treatment of bifurcated vessels and lesions.

Myler, et al., U.S. Pat. No. 5,474,563, discloses a cardiovascular stent and retrieval apparatus, whereby the stent may be inserted and retrieved through the intermediary of a balloon system, which is deployed in the lumen or body vessel of a patient. As in the previously discussed patents, there is no disclosure of the inventive type of stent and balloon delivery system, which is adapted for the treatment of bifurcated vessels and lesions.

Eury, U.S. Pat. No. 5,443,458, discloses a multilayered biodegradable stent and method of manufacture thereof, and does not concern itself with a novel stent and balloon delivery system employed for the treatment of bifurcated vessels and lesions as is disclosed by the present invention.

### SUMMARY OF THE INVENTION

Accordingly, pursuant to the invention, there is provided a unique and sophisticated advantageous design for a stent and integrated balloon stent delivery system enabling the conveyance to and deployment of the stent at a particular site in the lumen or body vessel of a patient. In particular, such as is contemplated for the treatment of a bifurcated vessel or lesion, there is provided a structure which incorporates a specialized stent and a side hole or aperture in the center portion of a balloon or by means of an integrated slit-tube embedded on a balloon surface, and/or along the shaft of a balloon catheter, in the form of either a separate structure or as an integrated delivery system for dilatation and/or placement of the stent or plurality of stents, while concurrently maintaining the patency of and access to both branches of the bifurcated vessel branch points. Although, especially directed to the treatment of bifurcated vessels or lesions, the inventive structure is not limited thereto.

Pursuant to the invention, the problems and technological challenges, which are set forth hereinabove and which are encountered in the current state-of-the-art are fully obviated or at the very least, extensively ameliorated, inasmuch as the novel stent and balloon delivery system, and the methodology of use thereof, clearly address all of the design constraints and issues encountered in the technology.

Accordingly, it is an object of the present invention to provide for a novel stent and balloon delivery system which is designed for, but not limited to the treatment of bifurcated vessels and lesions in the body of a patient.

Another object of the present invention is to provide a unique stent and balloon delivery system for the placement of a stent at a site, which is adapted for the treatment of lesions in a body vessel or lumen.

Still another object of the present invention resides in the provision of a unique system of stents and balloon stent delivery systems, which are adapted to effectuate the treatment of bifurcated vessels and lesions, maintaining the patency and access to both branches of a vessel branch point or points.

Another object of the present invention is to provide for a method of utilizing the inventive stent and balloon delivery system in the treatment of obstructive lesions in a body vessel or lumen.

Another object of the invention resides in the provision of a method utilizing the inventive stent and balloon delivery system in the treatment of bifurcated vessels and lesions encountered in the body of a patient.

### BRIEF DESCRIPTION OF THE ACCOMPANYING DRAWINGS

Reference may now be made to the following detailed description of preferred embodiments of the invention, taken in conjunction with the accompanying drawings; in which:

FIG. 1A and 1B diagrammatically illustrate, respectively, side views in various axially rotated orientations of a stent, which is constructed pursuant to the invention;

FIG. 1C illustrates an enlarged fragmentary view of the center portion of the stent of FIGS. 1A and 1B showing the larger open cell area;

FIG. 2A illustrates diagrammatically, an axial sectional view of a first embodiment of a stent and balloon delivery system for the deployment of the stent at a site in the body of a patient.

FIG. 2B illustrates, on an enlarged scale, showing a transverse sectional view taken at line 2B-2B in FIG. 2A;

FIG. 2C illustrates an axial view of the balloon showing the deployment of the stent of FIGS. 2A and 2B;

FIG. 3A illustrates, in a view similar to FIG. 2A, a modified embodiment of the stent and balloon delivery system;

FIG. 3B illustrates an axial view of the positioning of the stent at the intended site thereof;

FIG. 3C illustrates a transverse cross-sectional view of the stent and the balloon taken at line 3C-3C in FIG. 3B;

FIG. 3D illustrates a side view of a modified balloon with a slit-tube structure;

FIG. 3E illustrates a plan view of the structure of FIG. 3D;

FIG. 3F illustrates another modification in plan view with a catheter; and

FIGS. 4A through 4H illustrate sequential steps in the deployment of the bifurcated stent structure through the intermediary of the balloon delivery system.

### DETAILED DESCRIPTION OF THE INVENTION

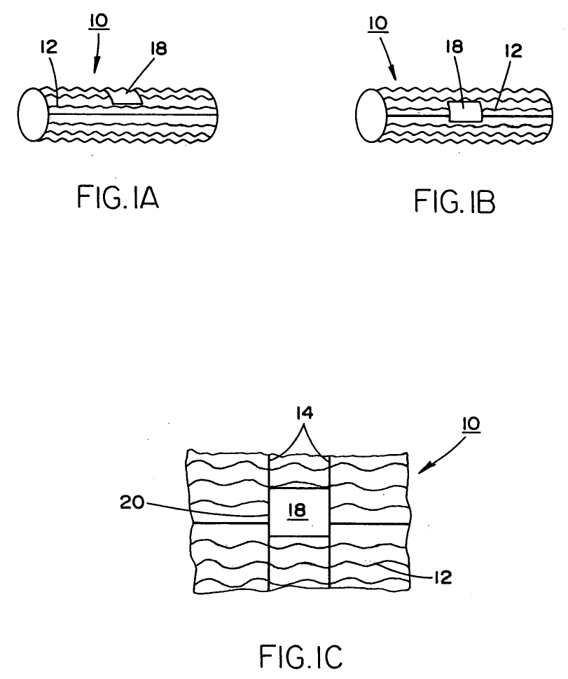

Referring in detail to the drawings, and particularly FIGS. 1A to 1C, there is illustrated a stent 10, which may include a wavylinear strut design 12 along the axial length thereof. The stent 10, which is radially expandable or contractable, includes a plurality of transverse strut connecting members 14, and may be constituted of either a medically or biocompatible plastic material or a surgical grade metal; for example, such as Nitinol (nickel-titanium alloy), as is well known in the stent implanting technology. Furthermore, the stent 10 may also be equipped, coated or impregnated with a drug or antibiotic dispensing system or release layer, as is known and presently employed in the medical art technology.

As illustrated in the drawings, a center region 18 of the stent 10 may possess a larger-sized open cell area 20, in effect, fewer of the struts 12, 14 in order to provide a larger opening 20, as shown in particular detail in FIG. 1C of the drawings, and as elucidated hereinbelow.

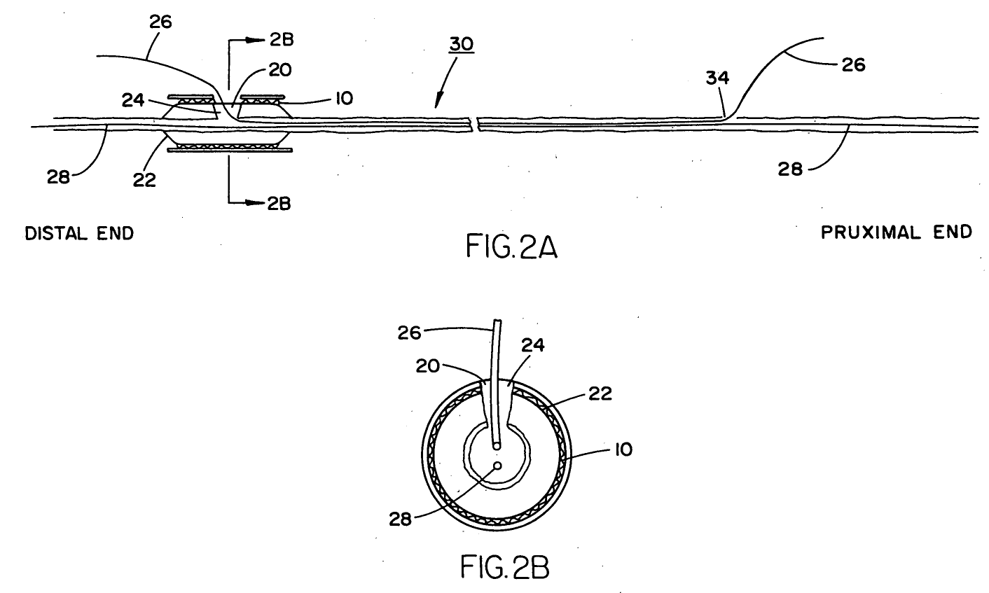

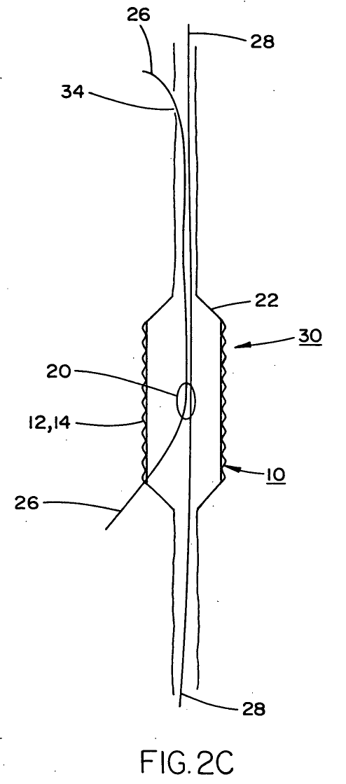

As indicated in the drawing FIGS. 2A through 2C, there is shown a composite or integrated stent and balloon delivery system 30 for deploying the stent 10 of FIGS. 1A through 1C in the lumen or body vessel of a patient.

In particular, the system 30, as illustrated in FIG. 2A, shows a balloon catheter 22 with either a central side guide wire exit location or site 24, facilitating a previously placed guide wire 26 in a main branch of a bifurcated vessel or lesion of a patient to be loaded backwards into the central side guide wire exit 24. A wire 28 at one distal end leads to a side branch and also wire 26 leads to the main branch in a bifurcated vessel or lumen of the patient through the side guide wire exit site formed in the stent 10 by the opening, which encompasses the periphery of the balloon 22.

At one end of the vessel, there is provided a dedicated exit point 34 for the second side guide wire 26, as shown in FIG. 2A of the drawings, whereas the central wire 28 is extended continuously through the main or central lumen of the patient.

As illustrated in FIG. 2A of the drawings, the second side exit wire 26 is shown exiting through the larger opening 20 in the stent 10, which encompasses the balloon 22, whereas the central wire 28 extends axially therethrough, as also shown by the cross-sectional view in FIG. 2B of the drawings.

As represented in FIG. 2C of the drawings, which shows a top plan view of the system 30, illustrating the larger aperture 20 formed in regions of the stent 10 through struts 14, 12, this shows the side or second side guide wire 26 being inserted therethrough and extending into the main branch of the patient's vessel, whereas the central wire 28 extends into the side branch.

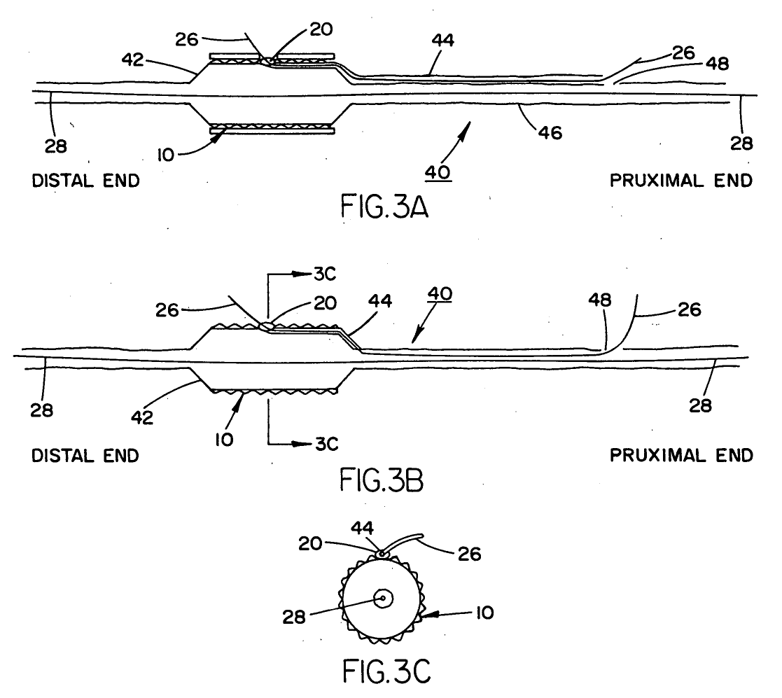

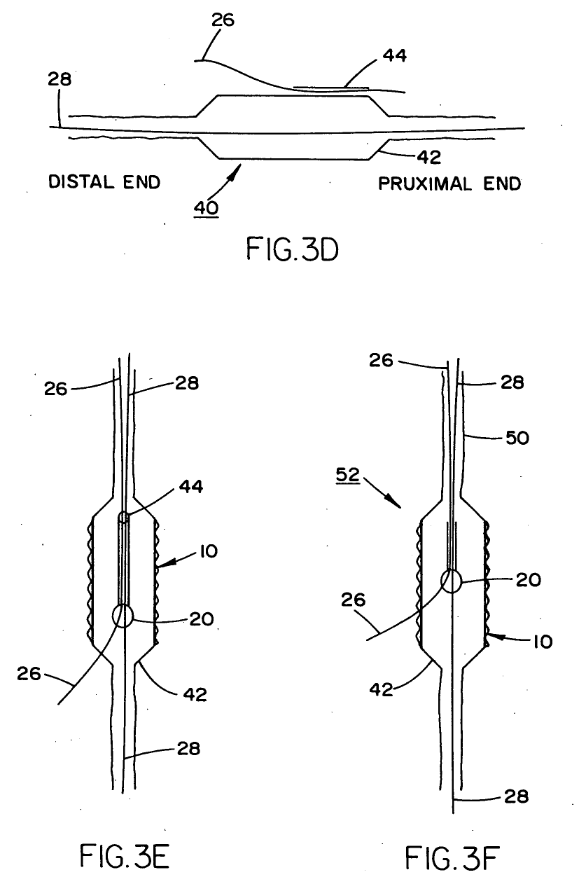

As shown in a modified version of the stent and balloon delivery system 40, referring to FIGS. 3A through 3F of the drawings, in that instance, the wire 26, which extends along to the main branch is directed through the larger aperture 20 in the stent 10, which encompasses the balloon 42, and then extends through a slit-tube 44 located at the outside surface 46 of the balloon 42, towards an exit site 48, whereas a central wire 28 extends through the balloon and the lumen of the patient arranged coaxially therewith.

As shown in FIG. 3B of the drawings, the wire 26, which leads to the main branch, extends through the larger sized opening 20 in the stent 10, whereas the central wire 28 leads to the side branch in a generally alternative arrangement of the wires.

As shown in FIG. 3C of the drawings, this illustrates in an enlarged transverse cross-sectional view, the central wire 28 extending axially and centrally through the balloon 42 stent 10 and the patient's lumen, whereas the other wire 26 extends through the slit-tube 44, as shown in FIG. 3A of the drawings, and through aperture 20 in stent 10 outwardly into the main branch of the vessel.

As indicated in FIG. 3C, this shows the central part of the tube 44 having been slit, enabling the wire 26 to exit along the slit portion and the site wire 26 being extended outwardly through the stent, whereas the other wire 28 extends centrally and axially through the balloon 42, as is also illustrated in FIG. 3D of the drawings.

Referring to FIG. 3E, this illustrates a top plan view showing the site wire 26 in the slit-tube 44 extending along the catheter, whereas the central wire 28 extends through the side branch of the bifurcated lumen, and the wire 26 from the slit-tube exits through the larger opening 20 in the center of the stent 10, so as to enter into the main branch of the bifurcated lumen of the patient.

As illustrated in FIG. 3F, in that instance, both wires 26, 28 are arranged in the shaft 50 of the balloon catheter 52, with one wire 26 extending upwardly through the large aperture in the stent to the main branch of the bifurcated vessel, whereas the other wire continues on through the balloon to the side branch.

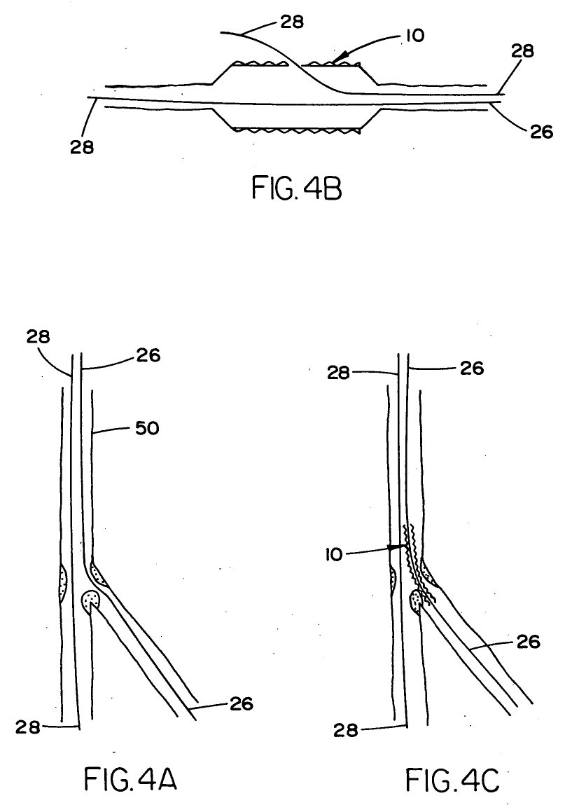

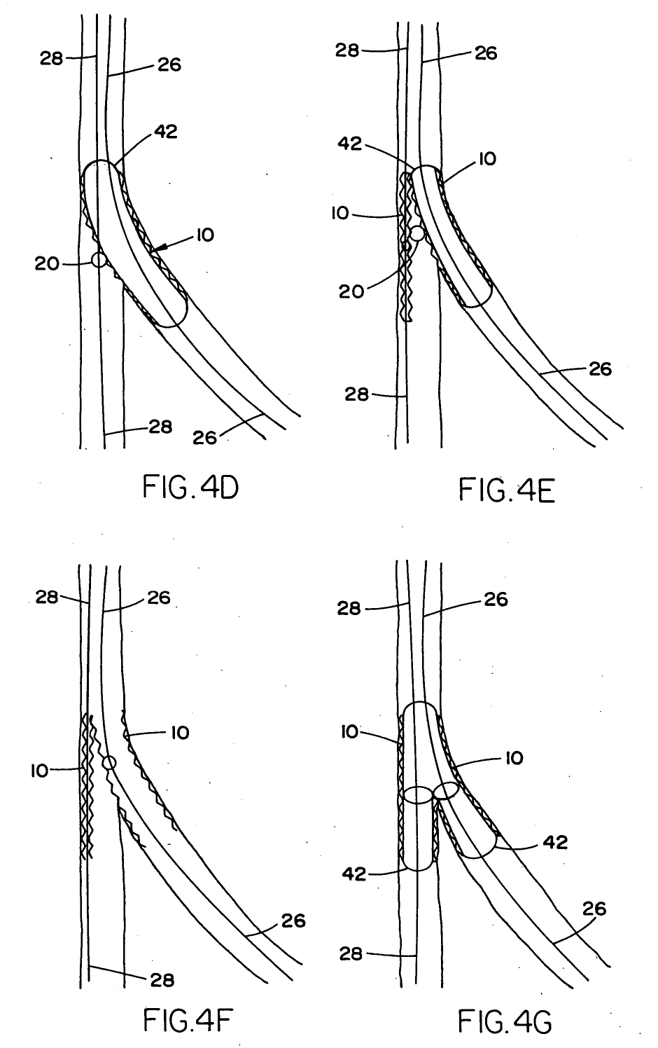

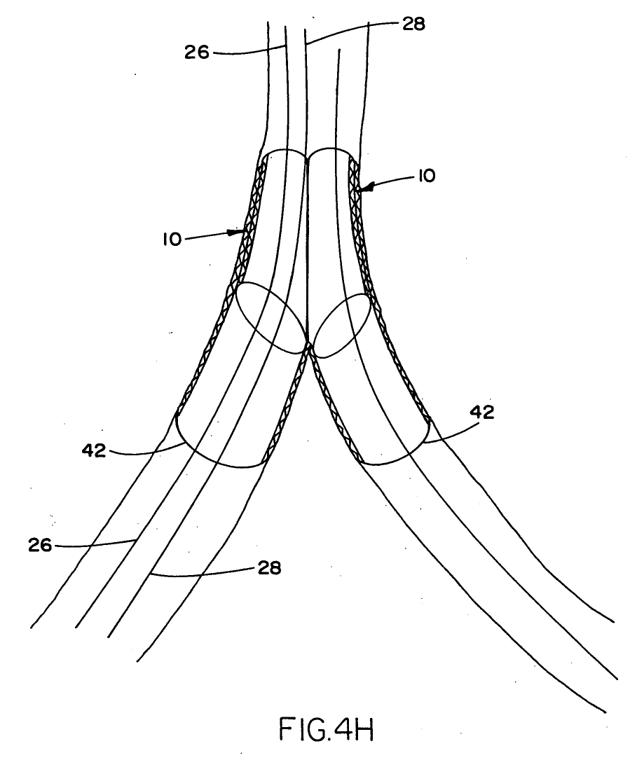

As illustrated in FIGS. 4A through 4H of the drawings, this illustrates the deployment steps in the method of positioning or siting of the stent 10 by means of the inventive stent deployment system (SDS).

In this instance, implementation of siting the stent is effected in substantially the following manner:
1) There is initiated the placement of the two separate guide wires 26, 28 into the main and side branches of bifurcated vessel, having reference to FIG. 4A.
2) The guide wire 26 in the side branch is loaded backwards into the central lumen, as represented in FIG. 4B.
3) The guide wire 28 in the main branch is loaded into the side-exit aperture/slit tube 44 and continued within the slit tube along the length or inside of the catheter shaft, referring to FIG. 4B.
4) The stent/balloon system is then advanced into the guide catheter with the two guide wires fixed in position.
5) The balloon/stent system is then advanced into the side branch until it is stopped by the guide wire, which was previously placed in the main branch and the position visually confirmed through fluoroscopy (FIG. 4C).
6) After carrying out of the visual confirmation, the balloon/stent is expanded and deployed in the side branch with the central larger open cell unit of the stent with the second guide wire residing in the main branch of the vessel of the patient (FIG. 4D).
7) The balloon in the expanded side branch is then deflated and withdrawn.
8) A similar balloon/stent is then loaded, this time with the central lumen loaded backwards using the guide wire in the main branch and the side-branch guide-wire loaded into the side exit or slit-tube, which is located on the side of the stent/balloon outer surface.
9) The second stent/balloon system is then advanced in a similar manner, as previously described in steps 4) and 5), except that the stent/balloon system is advanced into the main branch until it is stopped by the guide wire located in the side branch, and the position thereof visually confirmed by fluoroscopy (FIG. 4E).
10) After completing the visual confirmation, the balloon/stent is expanded and deployed in the main branch with the central larger open cell unit of the stent with the second guide wire centered in the side branch of the vessel (FIG. 4F). The balloon is then deflated and removed by being withdrawn (FIG. 4G).
11) Following inflation/deployment of the second stent/balloon system, two balloons with same or different sizes can then be advanced into place and simultaneous balloon inflation performed so as to maximize stent lumen and improve stent surface contact with the vessel wall. Herein, two stent layers are shown in the bifurcated vessel (FIG. 4H).
12) The balloon is then deflated and removed for angiographic examination of the treatment site.

From the foregoing, it becomes clearly obvious that the invention is directed at a unique stent and balloon stent delivery system, which is adapted particularly for but not limited to the treatment of bifurcated vessels and lesions in a patient, and also may be utilized for single lumens at various locations in a patient's body and for diverse treatments analogous to those currently employed in the technology.

While the invention has been particularly shown and described with respect to preferred embodiments thereof, it will be understood by those skilled in the art that the foregoing and other changes in form and details may be made therein without departing from the spirit and scope of the invention.

1. An expandable stent for implantation in a body vessel and for protecting the patency of the vessel; comprising:
a generally cylindrical structure having a plurality of axially extending struts at predetermined spacings relative to each other;
axially spaced struts radially extending about and interconnecting said struts; and
a central portion of said strut having an enlarged aperture formed between said axially extending struts to provide a larger maximal open cell areas upon expansion of said stent to facilitate passage therethrough of a side exit wire which is positionable in a main branch of aid vessel.

2. A stent as claimed in claim 1, wherein said axially extending struts have a variable configuration with a large inter-cellular spacing prior to strut expansion and said maximal open cell area subsequent to stent expansion.

3. A stent as claimed in claim 1, wherein said stent is constituted of a medical or surgical grade metal.

4. A stent as claimed in claim 3, wherein said metal comprises a nickel-titanium alloy.

5. A stent as claimed in claim 1, wherein said stent is constituted of a biocompatible plastic material.

6. A stent as claimed in claim 1, wherein said stent is provided with a drug release surface coating.

7. A stent as claimed in claim 1, wherein said enlarged aperture facilitates communication of said side exit wire with a side branch of said vessel so as to enable siting said stent for the treatment of bifurcated vessels and obstructive lesions.

8. A balloon catheter for delivering and deploying a stent in a body vessel for the treatment of obstructive lesions or the like, said stent encircling said balloon, said balloon catheter comprising a control or side exit site enabling a previously placed guide wire in a main branch of said vessel or lesion to extend into said control or side exit site into the shaft of said balloon catheter.

9. A balloon catheter as claimed in claim 8, wherein a second guide wire previously placed in a side branch of said vessel is extendable into a separate center lumen at an end of said balloon catheter for the treatment of bifurcated vessels and lesions.

10. A balloon catheter as claimed in claim 8, wherein said guide wire is extendable through an enlarged central or side exit aperture formed in the stent proximate the side exit site in said balloon.

11. A balloon catheter as claimed in claim 8, wherein a back end of said guide wire is directed to exit at selectively a mid, distal or proximal end of said balloon catheter shaft.

12. A balloon catheter as claimed in claim 10, wherein an integrated slit tube is fastened to the external surface of said balloon, said slit tube extending coaxially with said balloon from the middle of said surface and having an end terminating at a strut of said stent adjoining said enlarged side exit aperture in said strut.

13. A balloon catheter as claimed in claim 12, wherein said slit tube provides a guide path for said guide wire.

14. A balloon catheter as claimed in claim 12, wherein a further slit tube is positioned along said catheter for receiving a guide wire facilitating the advancement of said stent and balloon catheter.

15. A method for the delivery and deployment of stents at a site in bifurcated vessels or lesions encountered in a patient through the intermediary of an integrated stent and balloon catheter delivery system, said method comprising:
a. placing guide wires in, respectively, main and side branches of the bifurcated vessel;
b. loading the guide wire in the side branch backwards into a central lumen;
c. loading the guide wire in the main branch into a side exit hole in the catheter shaft and continuing the guide wire within the catheter shaft, or alternatively, guiding the wire within a slit tube extending externally along the lumen;
d. advancing the stent and balloon system into the catheter with said guide wires being fixed in place;
e. advancing the stent and balloon system into the side branch until stopped by the guide wire previously placed in the main branch, and confirming position by fluoroscopy;
f. expanding the balloon and stent while deployed in the side branch while the central larger aperture in the stent with the other guide wire is located in the main branch of the vessel; and
g. deflating and removing the balloon from the expanded side branch.

16. A method as claimed in claim 15, wherein deployment of a further stent and balloon catheter delivery system further comprises:
following steps a) through g) but reversing the placement of the guide wires between the main and side branches of the vessel or lesion, and wherein two said balloons of the same or selectively different sizes are utilized; and thereafter deflated and removed for angiographic examination of the treatment site.
